SEQUENCE LISTING (4 sequences)

SEQ ID NO: 1 - protein, 14 aa, Homo sapiens
  MISC_FEATURE (1)..(14): CDR3
ASSLGTGKTGYGYT

SEQ ID NO: 2 - protein, 13 aa, Homo sapiens
  MISC_FEATURE (1)..(13): CDR3
ASSASSSGANVLT

SEQ ID NO: 3 - protein, 14 aa, Homo sapiens
  MISC_FEATURE (1)..(14): CDR3
ASSPGSGTGNRLGF

SEQ ID NO: 4 - protein, 11 aa, Homo sapiens
  MISC_FEATURE (1)..(11): CDR3
  MISC_FEATURE (10)..(10): Xaa at position 10 may be any naturally-occurring amino acid
ASSLGSNTEXF

(19) United States
(12) Patent Application Publication
(10) Pub. No.: US 2022/0296642 A1
(43) Pub. Date: Sep. 22, 2022

(54) Methods of Making Therapeutic T Lymphocytes

(71) Applicant: NANTOMICS, LLC, Culver City, CA (US)

(72) Inventors: Andrew Nguyen, Culver City, CA (US); John Zachary Sanborn, Culver City, CA (US); Stephen Charles Benz, Culver City, CA (US); Sharooz Rabizadeh, Culver City, CA (US)

(21) Appl. No.: 17/294,891

(22) PCT Filed: Dec. 3, 2019
(86) PCT No.: PCT/US2019/064305

§ 371 Date: May 18, 2021

Related U.S. Application Data
(60) Provisional application No. 62/775,323, filed on Dec. 4, 2018.

(51) Int. Cl. A61K 35/17; C12N 5/0783; A61P 35/00

(52) U.S. Cl. CPC A61K 35/17; C12N 5/0636; A61P 35/00; C07K 2319/01

(57) ABSTRACT

Therapeutic T cells can be prepared from a population of TILs (tumor infiltrating lymphocytes) using tumor and patient-specific neoantigens expressed in antigen presenting cells to select for tumor reactive T cells. Selected tumor reactive T cells are then expanded and administered to the patient.

This application claims priority to our copending U.S. provisional patent application with the Ser. No. 62/775,323, which was filed Dec. 4, 2018, and which is incorporated by reference herein.

### SEQUENCE LISTING

The content of ASCII text file of the sequence listing named 102402.0082PCT_ST25, which is 2 kb in size was created on Nov. 27, 2019 and electronically submitted via EFS-Web along with the present application is incorporated by reference in its entirety.

### FIELD OF THE INVENTION

The field of the invention is computational analysis of omics data, particularly as it relates to identification of tumor-associated antigens that can be targeted by tumor-infiltrating lymphocytes in breast cancer patients.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Recent studies have demonstrated that the number of tumor infiltrating lymphocytes (TILs) positively correlates with outcome and response to chemotherapy in patients with Her2+ and Triple-Negative Breast Cancer (TNBC). Furthermore, first studies of immune-checkpoint inhibitors showed promising results in those patients. However, the targets of those TILs remain unknown. As such, isolation and clonal propagation is problematic, and the possibility of numerous distinct antigens further compounds difficulties associated with isolation of tumor reactive T cells.

Therefore, there is still a need to provide improved methods and compositions for T cell based immune therapy of cancer.

### SUMMARY OF THE INVENTION

The inventive subject matter is directed to various compositions and methods for the identification and/or expansion of neoantigen-reactive T cells obtained from a population of tumor infiltrating leukocytes (TIL). Most preferably, the thusly obtained T cells are reactive against patient- and tumor-specific neoantigens and can be used as therapeutic lymphocytes in the treatment of cancer in the patient.

In one aspect of the inventive subject matter, the inventors contemplate a method of generating therapeutic T cells that includes a step of obtaining patient-specific omics data from a tumor tissue of a patient, a further step of obtaining patient-specific omics data from a matched normal tissue of the same patient, and yet another step of comparing the omics data from the tumor tissue and the matched normal tissue to identify a tumor-specific neoantigen. In yet another step, a recombinant antigen-presenting cell is generated that comprises a recombinant nucleic acid encoding the tumor-specific neoantigen. A plurality of T cells obtained from tumor-infiltrating leukocytes are then contacted with the recombinant antigen-presenting cell, and one or more T cells that express an activation marker (e.g., cytokine or a chemokine such as IFN-γ) upon contact with the recombinant antigen-presenting cell are isolated from the plurality of T cells to so obtain the therapeutic T cells. These cells may be further expanded before prior administration to the patient (which may be supplemented by administration of an immunestimulatory cytokine and/or a checkpoint inhibitor to the patient.

As will be appreciated, the plurality of T cells may be expanded before contacting the T cells with the recombinant antigen-presenting cell, and/or the isolated T cells may be clonally distinct. Where desired, the isolated T cells may be tested for specificity against the tumor-specific neoantigen.

In some embodiments, the patient-specific omics data is in a BAMBAM format, a SAMBAM format, a FASTQ format, or a FASTA format, and the patient-specific omics data from the tumor comprises mutation information, copy number information, insertion information, deletion information, orientation information and/or breakpoint information. Typically, but not necessarily, the tumor-specific neoantigen is further identified by ascertaining expression of the neoantigen, binding affinity (dissociation constant) of the neoantigen to a MHC complex of the patient of equal or less than 200 nM, and/or exclusion of an SNP-based neoantigen.

In further embodiments, the recombinant antigen-presenting cell is derived from an autologous or HLA matched antigen-presenting cell, and is preferably a dendritic cell of the patient. Where desired, the recombinant nucleic acid encoding the tumor-specific neoantigen may further encode at least a second tumor-specific neoantigen. Alternatively, or additionally, the recombinant nucleic acid encoding the tumor-specific neoantigen may also encode a costimulatory molecule, an immune stimulating cytokine or cytokine analog, an OX40 ligand or fusion protein comprising OX40 ligand, and/or CD40 ligand or fusion protein comprising CD40 ligand.

Therefore, viewed from a different perspective, the inventors also contemplate an isolated T cell that is produced as presented herein as well as a pharmaceutical composition comprising a pharmaceutically acceptable carrier in combination with a plurality of isolated T cell produced as presented herein. Most typically, the composition will be formulated for transfusion and comprise at least 10⁷ isolated T cells.

Various objects, features, aspects and advantages of the inventive subject matter will become more apparent from the following detailed description of preferred embodiments, along with the accompanying drawing.

### BRIEF DESCRIPTION OF THE DRAWING

FIG. 1 shows a diagram of an exemplary antigen classification.

FIG. 2 shows an exemplary workflow chart for generating T cells targeting tumor specific neoantigens.

FIG. 3 shows exemplary FACS results for tumor infiltrating lymphocytes.

FIG. 4 shows exemplary results illustrating percentage of T-cells in different types of breast cancer as measured by flow cytometry.

FIG. 5 shows an exemplary workflow chart for expanding T cells targeting tumor specific neoantigens.

FIG. 6 shows exemplary FACS results for activated tumor infiltrating leucocytes that were isolated by flowcytometric sorting of IFN-γ secreting cells.

FIG. 7 shows exemplary FACS results for activated tumor infiltrating leucocytes that were isolated based on expression of CD137 48 h after peptide stimulation.

FIG. 8 schematically illustrates antigen recognition by a T cell receptor and the structure of T cell receptor.

FIG. 9 shows exemplary sequences of the CDR3 portion of the TCR.

FIG. 10A shows exemplary results indicating that autologous EBV-LCL were retrovirally transduced with full length mutated (mut) and wildtype (wt) antigen RBMX to demonstrate processing and presentation of the endogenously expressed protein (dark). As positive controls peptide loaded EBV-LCL were used (light).

FIG. 10B shows exemplary results indicating that transduction of mutated RBMX into the HLA class II negative breast cancer cell line MCF-7 cell line led to T-cell recognition after IFN-γ induced upregulation of HLA class II.

FIG. 10C shows exemplary results indicating that HLA-DP 04:01 overexpression in MCF-7 cells also resulted in effective presentation of the mutated antigen.

FIG. 10D shows exemplary results indicating that cellular lysates of RBMX mut transduced MCF-7 cells loaded on autologous EBV-LCLs induced a specific T-cell response.

FIG. 11 shows exemplary results of IFN-γ expression in various T cell clones retested against autologous EBV-LCL with and without peptide pool.

FIG. 12 shows exemplary bar graph of IFN-γ expression in 3E1 T cell clone tested against individual peptides of the HLA class II peptide pool and reacted specifically against peptide 28, a peptide derived from RBMX protein.

FIG. 13 shows exemplary bar graphs of IFN-γ expression of two other P28 specific T-cell clones.

FIG. 14 shows exemplary results for titration of wildtype and mutated peptide 28.

FIG. 15 shows exemplary results for IFN-γ expression of in T cell clone with MHC-CLASS I or II blocking antibodies.

FIG. 16 shows exemplary results for IFN-γ expression of T cell clone with MHC class II restriction molecules.

FIG. 17 shows exemplary results for IFN-γ expression of T cell clone 1A35 recognizing peptide 28.

FIG. 18 shows exemplary results for IFN-γ expression of cell clone 1A35 recognizing peptide 28 in HLA-II restriction molecule.

FIG. 19 shows another exemplary sequence of the CDR3 portion of the TCR.

### DETAILED DESCRIPTION

The inventors have now discovered methods of generating therapeutic T cells that can elicit a specific cytotoxic immune response against tumor cells in a patient by isolating and optionally expanding T cells from TILs of a tumor biopsy or other tumor sample of the patient that respond to one or more tumor-specific neoantigens genuine to a patient. Most typically, the tumor-specific neoantigens will be identified in silico from omics data of a tumor sample of the patient, and responsiveness to the neoantigens will preferably (but not necessarily) use autologous antigen presenting cells. Viewed from a different perspective, contemplated methods can be performed entirely ex vivo/in vitro using as little as omics data from a tumor sample and matched normal sample and a previously obtained blood sample.

For example, in one aspect of the inventive subject matter, the inventors contemplate a method of generating therapeutic T cells that includes a step of obtaining patient-specific omics data from a tumor tissue of a patient, a further step of obtaining patient-specific omics data from a matched normal tissue of the same patient, and yet another step of comparing the omics data from the tumor tissue and the matched normal tissue to identify a tumor-specific neoantigen. In yet another step, a recombinant antigen-presenting cell is generated that comprises a recombinant nucleic acid encoding the tumor-specific neoantigen. A plurality of T cells obtained from tumor-infiltrating leukocytes are then contacted with the recombinant antigen-presenting cell, and one or more T cells that express an activation marker (e.g., cytokine or a chemokine such as IFN-γ) upon contact with the recombinant antigen-presenting cell are isolated from the plurality of T cells to so obtain the therapeutic T cells, which may be further expanded.

As used herein, the term “tumor” refers to, and is interchangeably used with one or more cancer cells, cancer tissues (including metastases), malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use.

Neoantigens can be characterized as expressed random mutations in tumor cells that created unique and tumor specific antigens. Therefore, viewed from a different perspective, neoantigens may be identified by considering the type (e.g., deletion, insertion, transversion, transition, translocation) and impact of the mutation (e.g., non-sense, missense, frame shift, etc.), which may as such serve as a first content filter through which silent and other non-relevant (e.g., non-expressed) mutations are eliminated. It should further be appreciated that neoantigen sequences can be defined as sequence stretches with relatively short length (e.g., 7-11 mers) wherein such stretches will include the change(s) in the amino acid sequences. Most typically, the changed amino acid will be at or near the central amino acid position. For example, a typical neoantigen may have the structure of A₄-N-A₄, or A₃-N-A₅, or A₂-N-A₇, or A₅-N-A₃, or A₇-N-A₂, where A is a proteinogenic amino acid and N is a changed amino acid (relative to wild type or relative to matched normal). For example, neoantigen sequences as contemplated herein include sequence stretches with relatively short length (e.g., 5-30 mers, more typically 7-11 mers, or 12-25 mers) wherein such stretches include the change(s) in the amino acid sequences.

Thus, it should be appreciated that a single amino acid change may be present in numerous neoantigen sequences that include the changed amino acid, depending on the position of the changed amino acid. Advantageously, such sequence variability allows for multiple choices of neoantigens and so increases the number of potentially useful targets that can then be selected on the basis of one or more desirable traits (e.g., highest affinity to a patient HLA-type, highest structural stability, etc.). Most typically, neoantigens will be calculated to have a length of between 2-50 amino acids, more typically between 5-30 amino acids, and most typically between 9-15 amino acids, with a changed amino acid preferably centrally located or otherwise situated in a manner that improves its binding to MHC. For example, where the epitope is to be presented by the MHC-I complex, a typical neoantigen length will be about 8-11 amino acids, while the typical neoantigen length for presentation via MHC-II complex will have a length of about 13-17 amino acids. As will be readily appreciated, since the position of the changed amino acid in the neoantigen may be other than central, the actual peptide sequence and with that actual topology of the neoantigen may vary considerably.

Of course, it should be appreciated that the identification or discovery of neoantigens may start with a variety of biological materials, including fresh biopsies, frozen or otherwise preserved tissue or cell samples, circulating tumor cells, exosomes, cell free circulating DNA and/or RNA, various body fluids (and especially blood), etc. Therefore, suitable methods of omics analysis include nucleic acid sequencing, and particularly NGS methods operating on DNA (e.g., Illumina sequencing, ion torrent sequencing, 454 pyrosequencing, nanopore sequencing, etc.), RNA sequencing (e.g., RNAseq, reverse transcription based sequencing, etc.), and protein sequencing or mass spectroscopy based sequencing (e.g., SRM, MRM, CRM, etc.). As such, the omics data may cover the whole genome, exome, transcriptome, or portions thereof.

As such, and particularly for nucleic acid based sequencing, it should be particularly recognized that high-throughput genome sequencing of a tumor tissue will allow for rapid identification of neoantigens. However, it must be appreciated that where the so obtained sequence information is compared against a standard reference, the normally occurring inter-patient variation (e.g., due to SNPs, short indels, different number of repeats, etc.) as well as heterozygosity will result in a relatively large number of potential false positive neoantigens. Notably, such inaccuracies can be eliminated where a tumor sample of a patient is compared against a matched normal (i.e., non-tumor) sample of the same patient. Moreover, sequences may be eliminated that include a change due to a SNP.

In one especially preferred aspect of the inventive subject matter, DNA analysis is performed by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAMBAM format, SAMBAM format, FASTQ format, or FASTA format. However, it is especially preferred that the data sets are provided in BAMBAM format or as BAMBAM diff objects (see e.g., US2012/0059670A1 and US2012/0066001A1). Moreover, it should be noted that the data sets are reflective of a tumor and a matched normal sample of the same patient to so obtain patient and tumor specific information. Thus, genetic germ line alterations not giving rise to the tumor (e.g., silent mutation, SNP, etc.) can be excluded. Of course, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

Likewise, the computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive neoantigens and significantly reduces demands on memory and computational resources.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

Viewed from a different perspective, a patient- and cancer-specific in silico collection of sequences can be established that have a predetermined length of between 5 and 25 amino acids and include at least one changed amino acid. Such collection will typically include for each changed amino acid at least two, at least three, at least four, at least five, or at least six members in which the position of the changed amino acid is not identical. Such collection can then be used for further filtering (e.g., by sub-cellular location, transcription/expression level, MHC-I and/or II affinity, etc.) as is described in more detail below. For example, and using synchronous location guided analysis to tumor and matched normal sequence data, the inventors previously identified various cancer neoantigens from a variety of cancers and patients, including the following cancer types: BLCA, BRCA, CESC, COAD, DLBC, GBM, HNSC, KICH, KIRC, KIRP, LAML, LGG, LIHC, LUAD, LUSC, OV, PRAD, READ, SARC, SKCM, STAD, THCA, and UCEC. All neoantigen data can be found in US Patent Publication 2018/0141998, incorporated by reference herein.

Depending on the type and stage of the cancer, it should be noted that not all of the identified neoantigens will necessarily lead to a therapeutically equally effective reaction in a patient when checkpoint inhibitors are given to a patient. Indeed, it is well known in the art that only a fraction of neoantigens will generate an immune response. To increase likelihood of a therapeutically desirable response, the neoantigens can be further filtered. Of course, it should be appreciated that downstream analysis need not take into account silent mutations for the purpose of the methods presented herein. However, preferred mutation analyses will provide in addition to the type of mutation (e.g., deletion, insertion, transversion, transition, translocation) also information of the impact of the mutation (e.g., non-sense, missense, etc.) and may as such serve as a first content filter through which silent mutations are eliminated. For example, neoantigens can be selected for further consideration where the mutation is a frame-shift, non-sense, and/or missense mutation.

In a further filtering approach, neoantigens may also be subject to detailed analysis for sub-cellular location parameters. For example, neoantigen sequences may be selected for further consideration if the neoantigens are identified as having a membrane associated location (e.g., are located at the outside of a cell membrane of a cell) and/or if an in silico structural calculation confirms that the neoantigen is likely to be solvent exposed, or presents a structurally stable epitope (e.g., J Exp Med 2014), etc.

With respect to filtering neoantigens, it is generally contemplated that neoantigens are especially suitable for use herein where omics (or other) analysis reveals that the neoantigen is actually expressed. Identification of expression and expression level of a neoantigen can be performed in all manners known in the art and preferred methods include quantitative RNA (hnRNA or mRNA) analysis and/or quantitative proteomics analysis. Most typically, the threshold level for inclusion of neoantigens will be an expression level of at least 20%, at least 30%, at least 40%, or at least 50% of expression level of the corresponding matched normal sequence, thus ensuring that the (neo)epitope is at least potentially ‘visible’ to the immune system. Consequently, it is generally preferred that the omics analysis also includes an analysis of gene expression (transcriptomic analysis) to so help identify the level of expression for the gene with a mutation.

There are numerous methods of transcriptomic analysis known in the art, and all of the known methods are deemed suitable for use herein. For example, preferred materials include mRNA and primary transcripts (hnRNA), and RNA sequence information may be obtained from reverse transcribed polyAtRNA, which is in turn obtained from a tumor sample and a matched normal (healthy) sample of the same patient. Likewise, it should be noted that while polyAtRNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNA-seq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a cancer- and patient-specific mutation.

Similarly, proteomics analysis can be performed in numerous manners to ascertain actual translation of the RNA of the neoantigen, and all known manners of proteomics analysis are contemplated herein. However, particularly preferred proteomics methods include antibody-based methods and mass spectroscopic methods. Moreover, it should be noted that the proteomics analysis may not only provide qualitative or quantitative information about the protein per se, but may also include protein activity data where the protein has catalytic or other functional activity. One exemplary technique for conducting proteomic assays is described in U.S. Pat. No. 7,473,532, incorporated by reference herein. Further suitable methods of identification and even quantification of protein expression include various mass spectroscopic analyses (e.g., selective reaction monitoring (SRM), multiple reaction monitoring (MRM), and consecutive reaction monitoring (CRM)).

In yet another aspect of filtering, the neoantigens may be compared against a database that contains known human sequences (e.g., of the patient or a collection of patients) to so avoid use of a human-identical sequence. Moreover, filtering may also include removal of neoantigen sequences that are due to SNPs in the patient where the SNPs are present in both the tumor and the matched normal sequence. For example, dbSNP (The Single Nucleotide Polymorphism Database) is a free public archive for genetic variation within and across different species developed and hosted by the National Center for Biotechnology Information (NCBI) in collaboration with the National Human Genome Research Institute (NHGRI). Although the name of the database implies a collection of one class of polymorphisms only (single nucleotide polymorphisms (SNPs)), it in fact contains a relatively wide range of molecular variation: (1) SNPs, (2) short deletion and insertion polymorphisms (indels/DIPs), (3) microsatellite markers or short tandem repeats (STRs), (4) multinucleotide polymorphisms (MNPs), (5) heterozygous sequences, and (6) named variants. The dbSNP accepts apparently neutral polymorphisms, polymorphisms corresponding to known phenotypes, and regions of no variation.

Using such database and other filtering options as described above, the patient and tumor specific neoantigens may be filtered to remove those known sequences, yielding a sequence set with a plurality of neoantigen sequences having substantially reduced false positives.

Nevertheless, despite filtering, it should be recognized that not all neoantigens will be visible to the immune system as the neoantigens also need to be presented on the MHC complex of the patient. Indeed, only a fraction of the neoantigens will have sufficient affinity for presentation, and the large diversity of MHC complexes will preclude use of most, if not all, common neoantigens. Consequently, in the context of immune therapy it should thus be readily apparent that neoantigens will be more likely effective where the neoantigens are bound to and presented by the MHC complexes. Viewed from another perspective, treatment success with checkpoint inhibitors requires multiple neoantigens to be presented via the MHC complex in which the neoantigen must have a minimum affinity to the patient's HLA-type. Consequently, it should be appreciated that effective binding and presentation is a combined function of the sequence of the neoantigen and the particular HLA-type of a patient. Most typically, the HLA-type determination includes at least three MHC-I sub-types (e.g., HLA-A, HLA-B, HLA-C) and at least three MHC-II sub-types (e.g., HLA-DP, HLA-DQ, HLA-DR), preferably with each subtype being determined to at least 4-digit depth. However, greater depth (e.g., 6 digit, 8 digit) is also contemplated herein.

Once the HLA-type of the patient is ascertained (using e.g., known antibody-based chemistry or in silico determination), a structural solution for the HLA-type is calculated or obtained from a database, which is then used in a docking model in silico to determine binding affinity of the (typically filtered) neoantigen to the HLA structural solution. As will be further discussed below, suitable systems for determination of binding affinities include the NetMHC platform (see e.g., Nucleic Acids Res. 2008 Jul. 1; 36(Web Server issue): W509—W512.). Neoantigens with high affinity (e.g., having a K_{D} of less than 200 nM, less than 100 nM, less than 75 nM, less than 50 nM) for a previously determined HLA-type are then selected for therapy creation, along with the knowledge of the MHC-I/II subtype.

HLA determination can be performed using various methods in wet-chemistry that are well known in the art, and all of these methods are deemed suitable for use herein. However, in especially preferred methods, the HLA-type can also be predicted from omics data in silico using a reference sequence containing most or all of the known and/or common HLA-types as is shown in more detail below.

For example, in one preferred method according to the inventive subject matter, a relatively large number of patient sequence reads mapping to chromosome 6p21.3 (or any other location near/at which HLA alleles are found) is provided by a database or sequencing machine. Most typically the sequence reads will have a length of about 100-300 bases and comprise metadata, including read quality, alignment information, orientation, location, etc. For example, suitable formats include SAM, BAM, FASTA, GAR, etc. While not limiting to the inventive subject matter, it is generally preferred that the patient sequence reads provide a depth of coverage of at least 5×, more typically at least 10×, even more typically at least 20×, and most typically at least 30×.

In addition to the patient sequence reads, contemplated methods further employ one or more reference sequences that include a plurality of sequences of known and distinct HLA alleles. For example, a typical reference sequence may be a synthetic (without corresponding human or other mammalian counterpart) sequence that includes sequence segments of at least one HLA-type with multiple HLA-alleles of that HLA-type. For example, suitable reference sequences include a collection of known genomic sequences for at least 50 different alleles of HLA-A. Alternatively, or additionally, the reference sequence may also include a collection of known RNA sequences for at least 50 different alleles of HLA-A. Of course, and as further discussed in more detail below, the reference sequence is not limited to 50 alleles of HLA-A, but may have alternative composition with respect to HLA-type and number/composition of alleles. Most typically, the reference sequence will be in a computer readable format and will be provided from a database or other data storage device. For example, suitable reference sequence formats include FASTA, FASTQ, EMBL, GCG, or GenBank format, and may be directly obtained or built from data of a public data repository (e.g., IMGT, the International ImMunoGeneTics information system, or The Allele Frequency Net Database, EUROSTAM, URL: www.allelefrequencies.net). Alternatively, the reference sequence may also be built from individual known HLA-alleles based on one or more predetermined criteria such as allele frequency, ethnic allele distribution, common or rare allele types, etc.

Using the reference sequence, the patient sequence reads can now be threaded through a de Bruijn graph to identify the alleles with the best fit. In this context, it should be noted that each individual carries two alleles for each HLA-type, and that these alleles may be very similar, or in some cases even identical. Such high degree of similarity poses a significant problem for traditional alignment schemes. The inventor has now discovered that the HLA alleles, and even very closely related alleles can be resolved using an approach in which the de Bruijn graph is constructed by decomposing a sequence read into relatively small k-mers (typically having a length of between 10-20 bases), and by implementing a weighted vote process in which each patient sequence read provides a vote (“quantitative read support”) for each of the alleles on the basis of k-mers of that sequence read that match the sequence of the allele. The cumulatively highest vote for an allele then indicates the most likely predicted HLA allele. In addition, it is generally preferred that each fragment that is a match to the allele is also used to calculate the overall coverage and depth of coverage for that allele.

Scoring may further be improved or refined as needed, especially where many of the top hits are similar (e.g., where a significant portion of their score comes from a highly shared set of k-mers). For example, score refinement may include a weighting scheme in which alleles that are substantially similar (e.g., >99%, or other predetermined value) to the current top hit are removed from future consideration. Counts for k-mers used by the current top hit are then re-weighted by a factor (e.g., 0.5), and the scores for each HLA allele are recalculated by summing these weighted counts. This selection process is repeated to find a new top hit. The accuracy of the method can be even further improved using RNA sequence data that allows identification of the alleles expressed by a tumor, which may sometimes be just 1 of the 2 alleles present in the DNA. In further advantageous aspects of contemplated systems and methods, DNA or RNA, or a combination of both DNA and RNA can be processed to make HLA predictions that are highly accurate and can be derived from tumor or blood DNA or RNA. Further aspects, suitable methods and considerations for high-accuracy in silico HLA typing are described in US Patent Application 20180237949, incorporated by reference herein.

Once patient and tumor specific neoantigens and HLA-type are identified, further computational analysis can be performed by docking neoantigens to the HLA and determining best binders (e.g., lowest KD, for example, less than 500 nM, or less than 250 nM, or less than 150 nM, or less than 50 nM), for example, using NetMHC. It should be appreciated that such approach will not only identify specific neoantigens that are genuine to the patient and tumor, but also those neoantigens that are most likely to be presented on a cell and as such most likely to elicit an immune response with therapeutic effect. Of course, it should also be appreciated that thusly identified HLA-matched neoantigens can be biochemically validated in vitro prior to inclusion of the nucleic acid encoding the epitope as payload into the virus as is further discussed below.

Of course, it should be appreciated that matching of the patient's HLA-type to the patient- and cancer-specific neoantigen can be done using systems other than NetMHC, and suitable systems include NetMHC II, NetMHCpan, IEDB Analysis Resource (URL immuneepitope.org), RankPep, PREDEP, SVMHC, Epipredict, HLABinding, and others (see e.g., J Immunol Methods 2011;374:1-4). In calculating the highest affinity, it should be noted that the collection of neoantigen sequences in which the position of the altered amino acid is moved (supra) can be used. Alternatively, or additionally, modifications to the neoantigens may be implemented by adding N- and/or C-terminal modifications to further increase binding of the expressed neoantigen to the patient's HLA-type. Thus, neoantigens may be native as identified or further modified to better match a particular HLA-type. Moreover, where desired, binding of corresponding wildtype sequences (i.e., neoantigen sequence without amino acid change) can be calculated to ensure high differential affinities. For example, especially preferred high differential affinities in MHC binding between the neoantigen and its corresponding wildtype sequence are at least 2-fold, at least 5-fold, at least 10-fold, at least 100-fold, at least 500-fold, at least 1000-fold, etc.).

Thusly identified tumor-specific neoantigen sequence(s) can be cloned as one or more recombinant DNA sequences(s) into a recombinant expression vector that is expressable in a mammalian cell, preferably in human antigen presenting cells, and even more preferably the autologous antigen presenting cells (e.g., dendritic cells), which can be obtained from the patient's whole blood. As will be readily appreciated, the neoantigen sequences will typically have a length of at least nine amino acids, or at least 12 amino acids, or at least 15 amino acids, or at least 20 amino acids, or at least 30 amino acids, and may even be expressed as the full-length protein. Such antigen presenting cells can be further propagated to increase the number of available cells. As will be readily appreciated, any suitable method of introducing the recombinant nucleic acid having a sequence encoding the tumor-specific neoantigen are contemplated, including transfection, viral delivery, etc. In further contemplated aspects, and particularly where multiple neoantigens are used, the antigens may be arranged in a single polypeptide chain, typically with a flexible peptide linker between two neoantigens. Such arrangement advantageously increases the likelihood of antigen presentation that will activate previously isolated T cells in a population of TILs.

In addition, contemplated recombinant nucleic acids may also include further sequences that encode for a protein that enhances T cell stimulation. For example, suitable proteins include a costimulatory molecule (e.g., B7-1, B7-2, etc.), an immune stimulating cytokine (e.g., IL2, IL15, etc.) or cytokine analog (e.g., ALT-803), an OX40 ligand or fusion protein comprising OX40 ligand (e.g., OX40 receptor/OX40 ligand fusion), and/or CD40 ligand or fusion protein comprising CD40 ligand (e.g., CD40 receptor/CD40 ligand fusion). Consequently, and based on the above, it should be appreciated that autologous or HLA-compatible antigen presenting cells can be prepared that express one or more patient- and tumor-specific neoantigens that that can productively engage with T cells from a TIL population to activate one or more T cells based on the patient- and tumor-specific neoantigens.

Consequently, so generated recombinant antigen presenting cells (expressing the neoantigen) can be further contacted with autologous or HLA compatible T cells to identify T cells that are reactive to the recombinant antigen presenting cells. While any T cells that are suitable to react with the antigen presenting cells expressing the tumor-specific neoantigen are contemplated, preferred T cells includes patient's autologous T cells, more preferably, T cells in a population of TILs obtained from the tumor tissue (e.g., via biopsy, etc.). It is contemplated that a portion of T cell populations that has T cell receptors recognizing the tumor-specific neoantigen would be activated by contacting the antigen presenting cells expressing the tumor-specific neoantigen. Of course, it should be noted that the TILs and/or T cells in the TILs can be expanded before contacting the T cells with the antigen presenting cells, and all known manners of in vitro TIL/T cell expansion are deemed suitable for use herein. Consequently, such activation can be detected or quantified by measuring one or more activation markers (e.g., cytokine and/or chemokine) expressed and/or released by the T cell upon activation. Such cytokine and/or chemokine may include IL-2, IL-12, IL-17, gamma interferon, granulocyte-macrophage colony-stimulating factor.

The inventors further contemplate that T cells activated by the recombinant antigen presenting cells can be isolated and optionally further expanded to increase the population of T cells specifically recognizing tumor-specific neoantigen. Any suitable methods of expanding the T cells can be contemplated, including stimulation of isolated T cells with anti-CD3 antibodies and activation with IL-2 (e.g., 10-20 U/ml) for 3 days, for 7 days, for 14 days, etc. Optionally, such isolated (and optionally expanded) T cells can be further tested for specificity to the tumor-specific neoantigen by re-contacting the antigen presenting cells expressing the tumor-specific neoantigen.

In some embodiments, such isolated (and optionally expanded) T cells can be further formulated in a pharmaceutically acceptable carrier (e.g., for injection, etc.) and administered to the patient to treat the tumor. Without wishing to be bound by any specific theory, the inventors contemplate that such T cells recognizing the tumor-specific neoantigen on a tumor can specifically target the tumor cells in vivo to elicit a therapeutically effective immune response and increase the efficiency and specificity of immune therapy. As will be readily recognized in the art, further immune stimulation (e.g., with an immune stimulatory cytokine and/or checkpoint inhibitor) may enhance the therapeutic effect of the T cells.

### Examples

As shown in FIG. 1, human tissue may carry various types of antigens. Among those, the inventors contemplate that tumor-specific neoantigens, which are not self-antigens, can provide a high-avidity, tumor-specific T cell response.

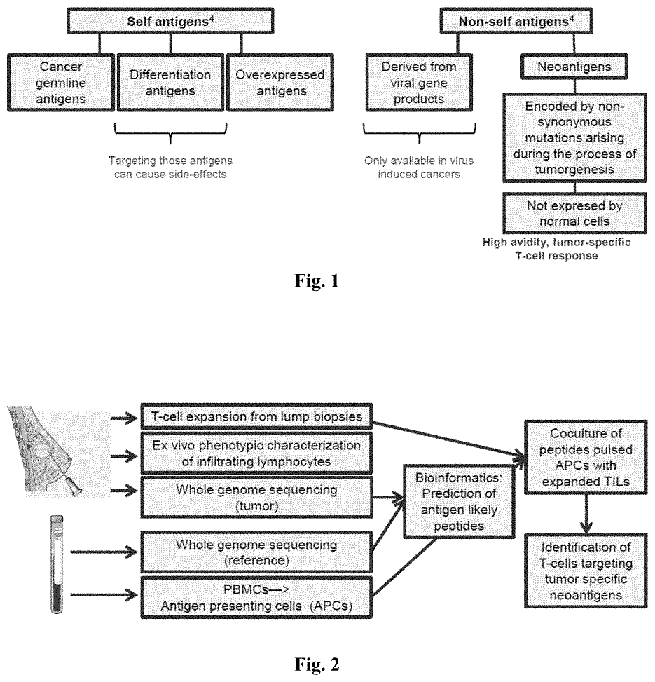

In an exemplary workflow, as illustrated in FIG. 2, tumor-infiltrating leukocytes (TILs) from breast cancer biopsies taken at the time point of diagnosis were expanded by unspecific stimulation. Additionally, the inventors used the Gentle Macs Dissociator in combination with flow cytometry to investigate the number of TILs in the tumor tissue. Furthermore, the inventors performed whole-genome sequencing of the tumor tissue and as reference autologous white blood cells to determine patient- and tumor-specific mutations. Here, location guided synchronous alignment led to the detection of mutations leading to a non-synonymous amino acid change. These mutations were analyzed for RNA expression of the encoding gene as well as to determine potential neoantigens. Neoantigens were evaluated for their potential binding to the patient's specific HLA molecules with a cut-off Kd of equal or less than 200 nM. Peptides for potential neoantigens were then synthesized, loaded onto autologous antigen presenting cells (APCs) and co-cultured with TILs. All IFNγ producing T-cells were clonally expanded and re-tested for peptide specificity to identify neoantigen specific T-cell clones.

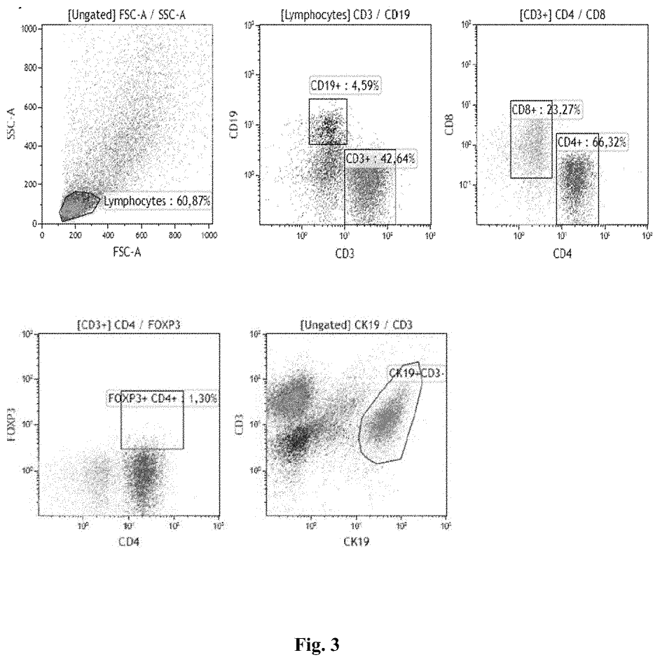

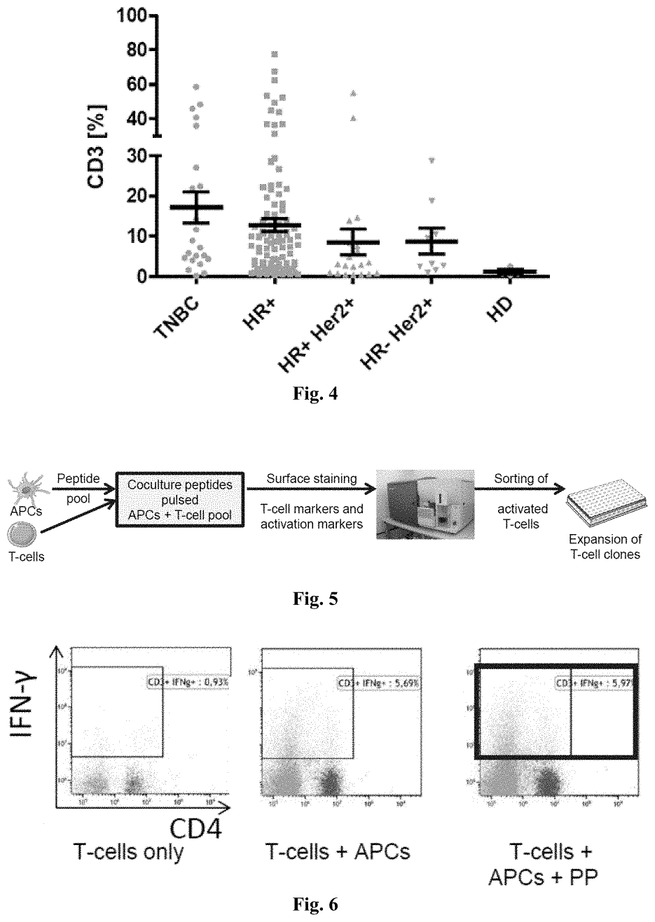

FIG. 3 shows FACS data of examples of an ex vivo phenotypic characterization of infiltrating lymphocytes (e.g., CD19+, CD4+, CD8+, etc.) in a breast lump biopsy after GentleMACS dissociation, and FIG. 4 shows percentages of CD3+ T-cells in different types of breast cancer as measured by flow cytometry. As can be seen, there are higher frequencies of TILs in TNBC as compared to hormone receptor (HR) or Her2 positive types of breast cancer and healthy donors (HD).

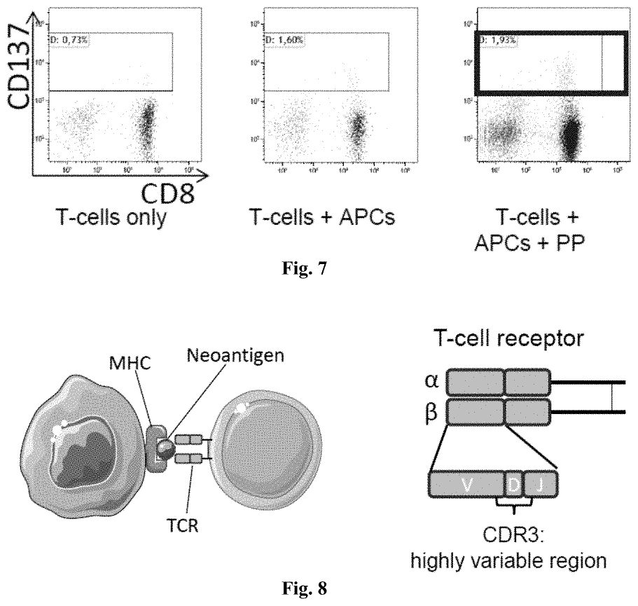

FIG. 5 shows an exemplary work flow for selected experiments below in which the antigen presenting cells (APCs) that express tumor-specific neoantigens (by peptide-pulse protocol) and T cells were co-cultured. T cells were further sorted via FACS and expanded. More specifically, FIG. 6 shows the results for isolation of activated T cells by FACS of IFN-γ-secreting cells. Note that the number of activated CD4+ T cells could be increased by contacting the antigen presenting cells (APCs). FIG. 7 shows isolation of activated T cells based on the expression of CD137 48 hours after stimulation. Activation of CD8+ T cells was increased, but in less extent, compared to CD4+ T cells.

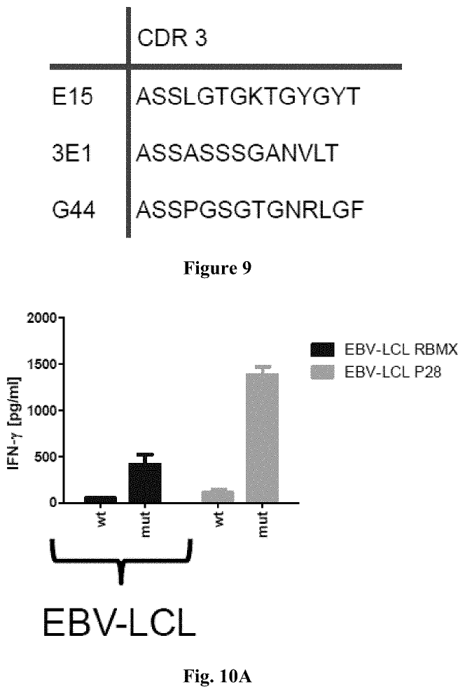

From the isolated T cells, the inventors identified sequence of CDR3 portions (as illustrated in FIG. 8). Based on CDR3 sequencing it was shown that the three isolated T-cell clones represented individual clones demonstrating polyclonality of the T-cell response. FIG. 9 shows amino acid sequence CDR3 portion of three T cell clones identified as SEQ ID NO:1, SEQ ID NO:2, and SEQ ID NO3.

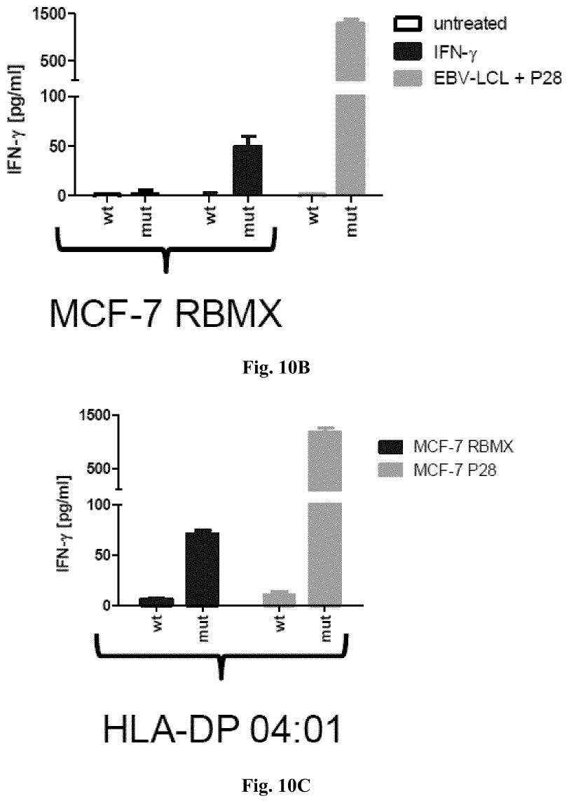

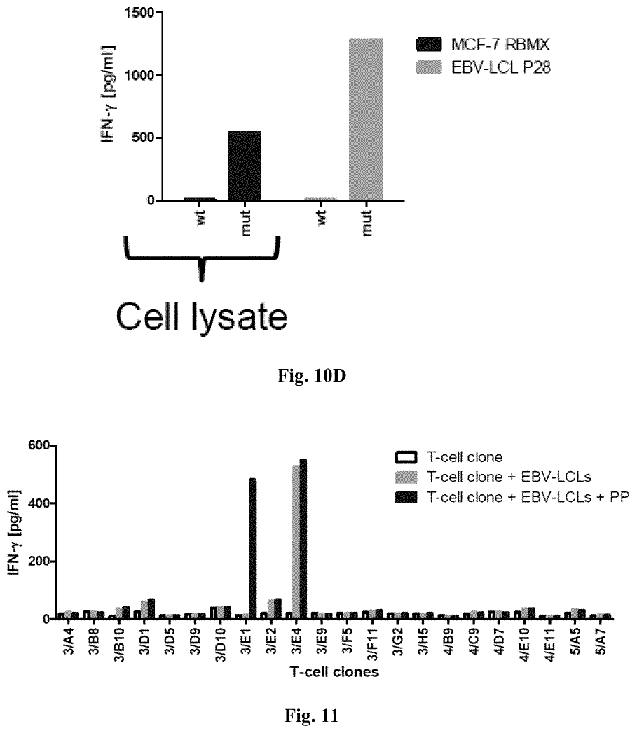

Autologous EBV-LCL (EBV infected lymphoblastoid cell line) cells were retrovirally transduced with the full length mutated (mut) and wildtype (wt) antigen RBMX, and so prepared cells were contacted with reactive T cells. FIG. 10A demonstrates processing and presentation of the endogenously expressed protein (blue). As positive controls peptide loaded EBV-LCL were used (grey). FIG. 10B shows that transduction of mutated RBMX into the HLA class II negative breast cancer cell line MCF-7 cell line led to T-cell recognition after IFN-γ induced upregulation of HLA class II. As shown in FIG. 10C, HLA-DP 04:01 overexpression in MCF-7 cells also resulted in effective presentation of the mutated antigen. Further, as shown in FIG. 10D, cellular lysates of RBMX mut transduced MCF-7 cells loaded on autologous EBV-LCLs induced a specific T-cell response.

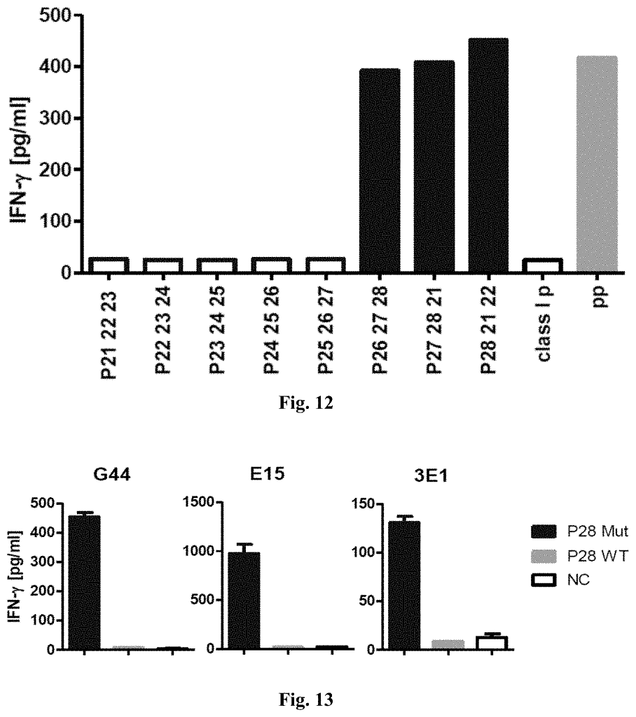

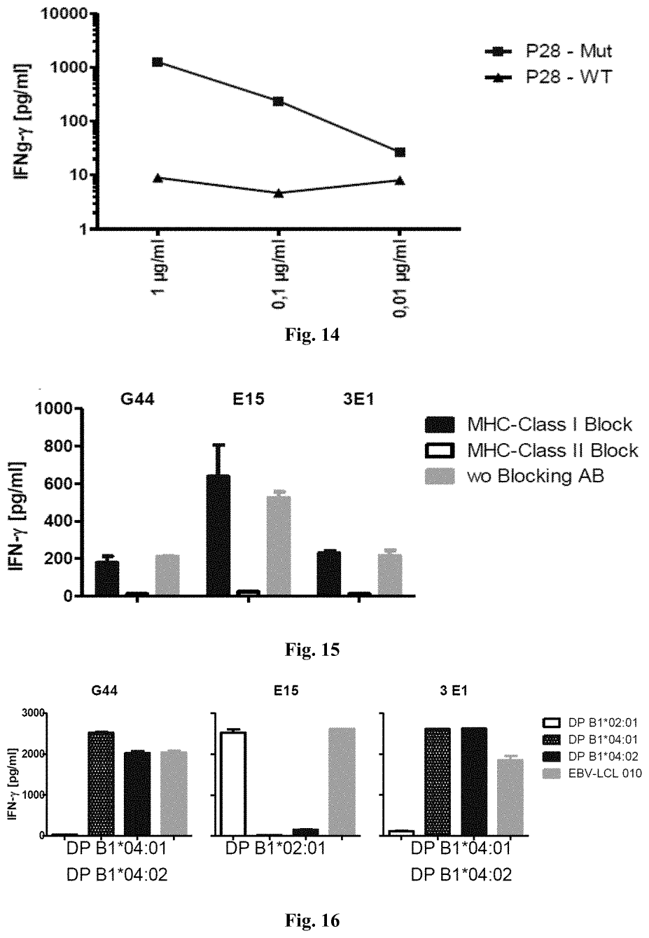

Expanded T-cell clones were then re-tested against autologous EBV-LCL with and without peptide pool in IFNγ ELISA. Among all clones, as shown in FIG. 11, clone 3E1 showed potential neoantigen (PP) specificity. Then, CD4+ T-cell clone 3E1 was tested against individual peptides of the HLA class II peptide pool. FIG. 12 shows that clone 3E1 reacted specifically against peptide 28, a peptide derived from RBMX protein. Further, as shown in FIG. 13, two other P28 specific T-cell clones were found among the TILs. All of them recognized the mutated P28 and not the wildtype counterpart. FIG. 14 illustrates titration of wildtype and mutated peptide 28.

The inventors further confirmed that such reaction is HLA-type specific. As shown in FIG. 15, MHC-Class II blocking antibodies blocked T-cell response in IFNγ ELISA confirming MHC-Class II restriction of the epitope. Further as shown in FIG. 16, HLA class II restriction molecules of the patient were retrovirally transduced into HeLa cells. Peptide 28 was presented in both HLA-DP restriction molecules of the patient. Two of the clones recognized the peptide in HLA-DPB1*0401 and one in HLA-DPB1*0201. The HLA-DPB1*0401 restricted T-cell clone also recognized the peptide in HLA-DPB1*0402. Autologous EBV-LCL served as positive control.

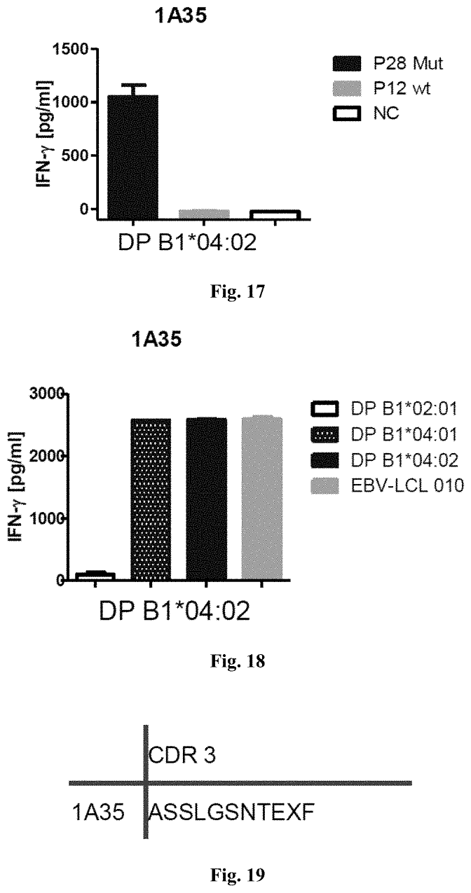

In addition to the biopsy obtained at the time point of initial diagnosis, the inventors also analyzed the resected tumor tissue after neoadjuvant therapy. FIG. 17 shows that a forth T-cell clone (1A35) recognizing peptide 28 could be identified. The 1A35 clone could recognize peptide 28 in HLA DP B1*04:01 and B1*04:02, as shown in FIG. 18, and FIG. 19 shows the sequence of CDR3 region (SEQ ID NO:4) of the 1A35 clone that is different as compared to the clone identified at initial diagnosis.

Thus, the inventor's flow cytometric analysis of the tumor biopsy for more than 300 patients showed higher frequencies of TILs in TNBC as compared to other types of breast cancer or patients without malignancy. Screening for neoantigen specific T-cells in one patient led to identification of three peptide-specific CD4+ T-cell clones isolated from Her2+ breast cancer tissue taken at the time point of diagnosis. All T-cell clones specifically recognized the same tumor-specific mutation and not the wildtype counterpart. Furthermore, the inventors demonstrated that these T-cell clones also recognized the endogenously expressed mutated antigen. This verified the ability of processing and presentation of the respective protein. Interestingly, the inventors could also isolate a T-cell clone recognizing the same neoantigen in the resected tumor tissue after neoadjuvant therapy. Based on CDR3 sequencing the inventors could prove that the four T-cell clones represented individual clones. This confirms the polyclonal nature of the immune response. Moreover, the inventors showed that the same neoantigen was presented in two different HLA restriction molecules of the patient with three of the clones recognizing it in HLA-DPB1*0401 and one in HLA-DPB1*0201. These results further underline the immunogenicity of this neoantigen.

In conclusion, the data demonstrates tumor-specificity of TILs in a patient with Her2+ breast cancer. Furthermore, the inventors show the feasibility to identify individual cancer specific T-cell targets in breast cancer patients. These results may contribute to the development of targeted patient-specific immunotherapies in the future.

The description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Moreover, all methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of generating therapeutic T cells, comprising:
obtaining patient-specific omics data from a tumor tissue of a patient, and obtaining patient-specific omics data from a matched normal tissue of the same patient, and comparing the omics data from the tumor tissue and the matched normal tissue to identify a tumor-specific neoantigen;
generating a recombinant antigen-presenting cell having a recombinant nucleic acid encoding the tumor-specific neoantigen;
contacting a plurality of T cells with the recombinant antigen-presenting cell, wherein the T cells are obtained from tumor-infiltrating leukocytes from the patient; and
isolating from the plurality of T cells one or more T cells that express an activation marker upon contact with the recombinant antigen-presenting cell to so obtain the therapeutic T cells.

2. The method of claim 1, wherein the patient-specific omics data is in a BAMBAM format, a SAMBAM format, a FASTQ format, or a FASTA format.

3. The method of claim 1, wherein the patient-specific omics data from the tumor comprises mutation information, copy number information, insertion information, deletion information, orientation information and/or breakpoint information.

4. The method of claim 1, wherein the tumor-specific neoantigen is further identified by at least one of ascertaining expression of the neoantigen, binding affinity of the neoantigen to a MHC complex of the patient of equal or less than 200 nM, and exclusion of an SNP-based neoantigen.

5. The method of claim 1, wherein the recombinant antigen-presenting cell is derived from an autologous or HLA matched antigen-presenting cell.

6. The method of claim 5, wherein the autologous recombinant antigen-presenting cell is a dendritic cell of the patient.

7. The method of claim 1, wherein the recombinant nucleic acid encoding the tumor-specific neoantigen further encodes at least a second tumor-specific neoantigen.

8. The method of claim 1, wherein the recombinant nucleic acid encoding the tumor-specific neoantigen further encodes a costimulatory molecule, an immune stimulating cytokine or cytokine analog, an OX40 ligand or fusion protein comprising OX40 ligand, and/or CD40 ligand or fusion protein comprising CD40 ligand.

9. The method of claim 1, wherein the plurality of T cells are expanded before the step of contacting the plurality of T cells with the recombinant antigen-presenting cell.

10. The method of claim 1, wherein the activation marker comprises a cytokine or a chemokine.

11. The method of claim 1, wherein the cytokine comprises IFN-γ.

12. The method of claim 1, wherein the isolated T cells are clonally distinct.

13. The method of claim 1, further comprising retesting the isolated T cells for specificity against the tumor-specific neoantigen.

14. The method of claim 1, further comprising expanding the isolated plurality of T cells before administering the expanded T cells to the patient.

15. The method of claim 14 further comprising a step of administering the expanded T cells to the patient.

16. The method of claim 15 further comprising a step of administering an immunestimulatory cytokine and/or a checkpoint inhibitor to the patient.

17. An isolated T cell produced by the method of claim 1.

18. A pharmaceutical composition comprising a pharmaceutically acceptable carrier in combination with a plurality of isolated T cell produced by the method of claim 1.

19. The composition of claim 19 wherein the isolated T cells are monoclonal.

20. The composition of claim 19 formulated for transfusion and comprising at least 10⁷ isolated T cells.
